(19) United States
(12) Patent Application Publication
(10) Pub. No.: US 2021/0345915 A1
(43) Pub. Date: Nov. 11, 2021

(54) Methods Circuits Devices Systems and Machine Executable Code for Glucose Monitoring Analysis and Remedy

(71) Applicant: CaloSense Ltd., Netanya (IL)

(72) Inventor: Nitzan Shenar, Netanya (IL)

(73) Assignee: CaloSense Ltd., Netanya (IL)

(21) Appl. No.: 17/315,325

(22) Filed: May 9, 2021

Related U.S. Application Data
(60) Provisional application No. 63/022,440, filed on May 9, 2020.

(51) Int. Cl. A61B 5/145; G16H 50/30; G16H 10/60; G16H 20/10

(52) U.S. Cl. CPC A61B 5/14532; G16H 20/10; G16H 10/60; G16H 50/30

(57) ABSTRACT

Disclosed are methods, circuits, devices, systems and functionally associated machine executable code for glucose monitoring, analysis and remedy. A subject glucose level baseline is calculated based on monitored subject glucose level readings collected by a non-invasive sensor assembly and subject mobile device sensors data. Newly received, monitored subject glucose level readings sets are compared to subject glucose level baseline values to detect anomalies Indications of a glucose level anomaly, are analyzed by reference of one or more subject behavioral or physiological conditions concurrent with the anomaly. Monitored subject behavioral and physiological conditions are analyzed to determine a representation of the subject over a multi-condition diabetic risk graph/map, subject feedback is generated based on the representation.

### RELATED APPLICATIONS SECTION

The present application claims priority from U.S. Provisional Patent Application No. 63/022,440, filed May 9, 2020, which application is hereby incorporated by reference in its entirety.

### FIELD OF THE INVENTION

The present invention generally relates to the fields of chronic disease prevention by lifestyle management. More specifically, the present invention relates to methods, circuits, devices, systems and machine executable code for glucose monitoring, analysis and remedy.

### BACKGROUND

Diabetes is one of the biggest global health crises of the 21st century, with an estimation of 1B diabetic & pre-diabetics globally. Only a change of lifestyle can reverse condition effectively. Pre-Ds are scared and confused but mostly feel helpless—and in 5-6 years 30% of them will develop diabetes. Estimating the total costs of diagnosed diabetics has risen to $327 billion in 2008, in the USA alone.

There remains a need, in the field of chronic disease prevention by lifestyle management, for solutions that may reverse prediabetes by a personalized & real-time intervention platform that manages lifestyle with data gathered via noninvasive sensors.

### SUMMARY OF THE INVENTION

Embodiments of the present invention include methods, circuits, devices, systems and machine executable code for glucose monitoring, analysis and remedy.

There may be provided, in accordance with some embodiments, a glucose monitoring, analysis and remedy system, including a glucose analysis sever/logic for generating a monitored subject glucose level baseline, also referred to herein as a ‘Personal Glucose Signature’ or PGS—representing the subject's common/usual glucose level values along the course of a day.

The subject glucose level baseline may be calculated based on median values, and/or other measure(s) of central tendency, of monitored subject glucose level readings, collected by a non-invasive sensor assembly and communicated through a monitored subject mobile device application, along with mobile device sensors data.

Newly received, monitored subject glucose level readings sets may be compared to subject glucose level baseline values sets relating to the same, or substantially the same, time of the day, to detect anomalies between the two value sets which are indicative of specifically characterized glucose level jumps/rises.

An indication of a glucose level anomaly, may be analyzed by reference of one or more subject behavioral or physiological conditions concurrent with the anomaly, wherein the behavioral or physiological conditions are derived/calculated/concluded based on readings/data from a combination of one or more of the assembly sensors and the mobile device sensors.

A monitored subject feedback, and/or an alert/notification, may be generated or selected at least partially based on the type, level and/or characteristics of the behavioral or physiological condition experienced by the monitored subject concurrently with the glucose level anomaly.

The behavioral or physiological conditions may, in accordance with some embodiments, include a combination of at least the following conditions and may be non-invasively and continuously/intermittently sensed/derived/calculated/concluded as follows:
Glucose Level—Using a Photoplethysmograph (PPG) sensor, Bioimpedance sensor, or any other glucose sensor or sensing technique.
Sleep—Based on a combination of an accelerometer (on sensor-composite, smart-watch, mobile device) readings and monitored subject heartbeat rate sensor readings.
Stress Level—Based on measured glucose level anomalies and disorders, Galvanic Skin Response (GSR) sensor, or other.
Body Mass Index (BMI)—Based on Bioimpedance sensor readings.
Physical Activity—Based on an accelerometer (on sensor-composite, smart-watch, mobile device) readings. May also be indicative on length, frequency, intensity, and type (e.g. Aerobic/Cardio, Anaerobic) and may be assisted by a GPS or other positioning sensor/technique.
Liver Glycogen—Deducted based on the detection (as described above) of a monitored subject which is: a sleep and thus not eating, not shown to be experiencing stress and has concurrently experienced a Glucose level rise/anomaly. Accordingly—if no food intake, stress, or physical activity is found to be associated with the detected glucose rise/anomaly—the remaining, deducted, glucose level effecting condition, is liver or cell glycogen being broken and glucose being released to bloodstream.
   Food Intake—analysis of glucose level anomalies behavior patterns—for example by using anomaly pattern data as training data for an AI/NN learning process, to later cluster/group similarly patterned anomalies—may be utilized for out-filtering of conditions not relevant, (e.g. not having the same/similar pattern, to a detected glucose anomaly. Accordingly, a given anomaly may be regarded as: non-activity, non-stress and non-liver-related—and hence, concluded to be food intake related.

Monitored subject assembly/mobile-device sensors data and subject behavioral or physiological condition derived therefrom, along with other provided subject specific information, may be analyzed over time to generate multiple subject vectors, each representing a subject's lifestyle (i.e. measured along time) score associated with another condition.

Multiple condition vectors scores of a given subject may be used to find/construct a position/shape representing the subject over a multi-condition diabetic risk graph/map, a general subject diabetic risk/tendency score may be generated based on the conditions scores.

Graph/map representations of multiple subjects' may be clustered, wherein graph/map representations, substantially similarly positioned/shaped over the graph/map, may be associated with a same cluster.

Clusters of higher and lower diabetic risk may be designated based on their associated subjects' general risk/tendency levels. The diabetic risk of a given subject may be predicted/forecasted based on the prior recorded movements, of former subject's-cluster members, towards/in-the-direction of either higher or lower risk/tendency clusters over the graph/map.

One or more specifically selected/constructed lifestyle recommendations, to efficiently/rapidly—while optionally also factoring subject recommendation selection/preference—mobilize/change, over time, the subject's graph/map representation towards/to a graph/map representation associated with a subjects' cluster of a lower diabetic risk score than the current monitored-subject, or monitored-subject cluster, risk score.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings:

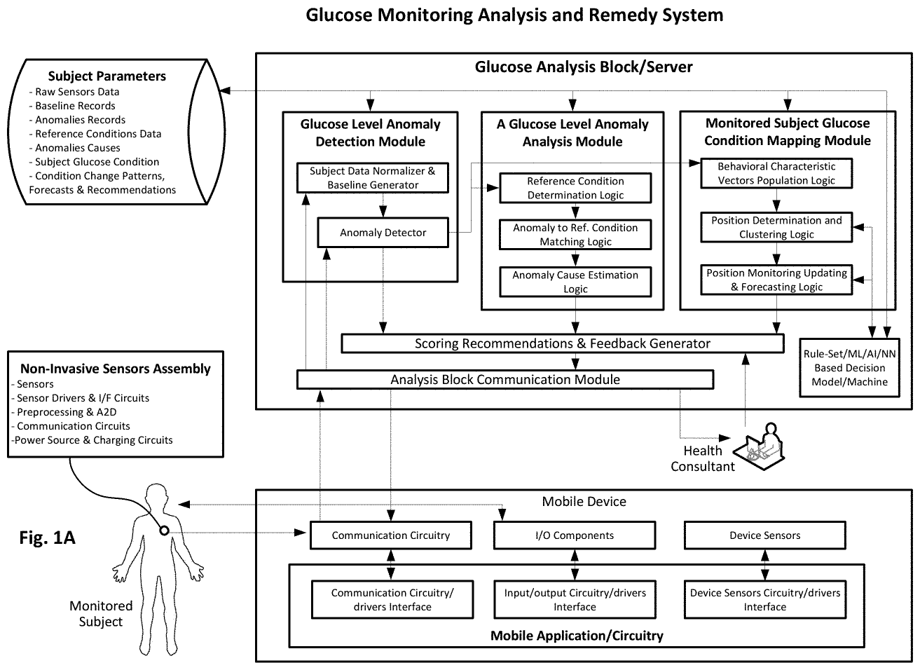

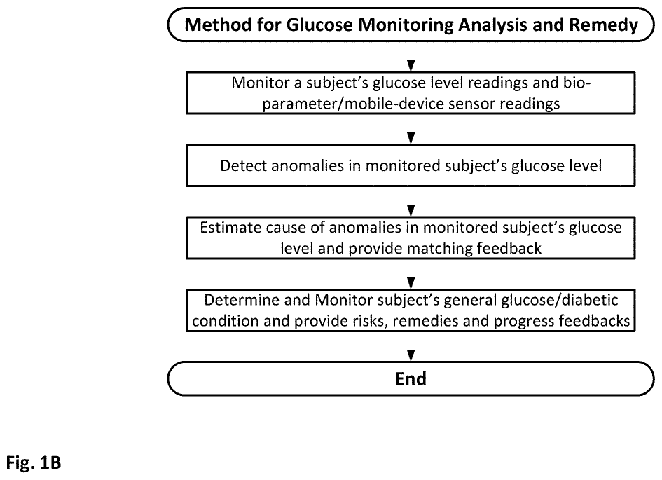

In FIG. 1A, there is shown a block diagram of an exemplary system for glucose monitoring, analysis, and remedy, including components thereof and interconnections there between, in accordance with some embodiments of the present invention;

In FIG. 1B, there is shown a flowchart of the main steps executed as part of an exemplary glucose monitoring, analysis, and remedy process, in accordance with some embodiments of the present invention;

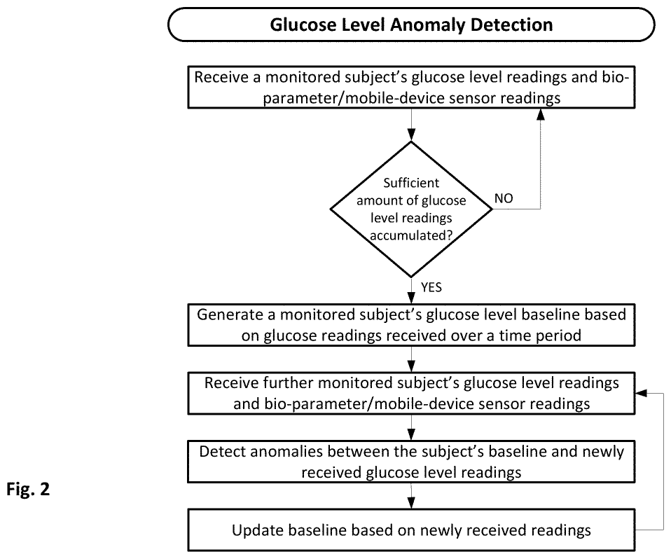

In FIG. 2, there is shown a flowchart of the main steps executed as part of an exemplary glucose level anomaly detection process, in accordance with some embodiments of the present invention;

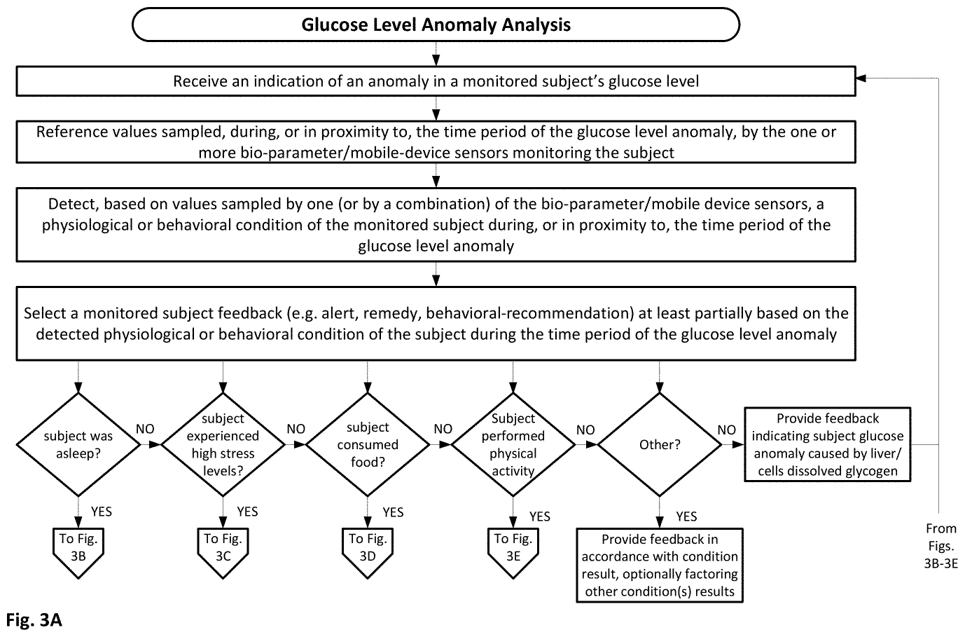

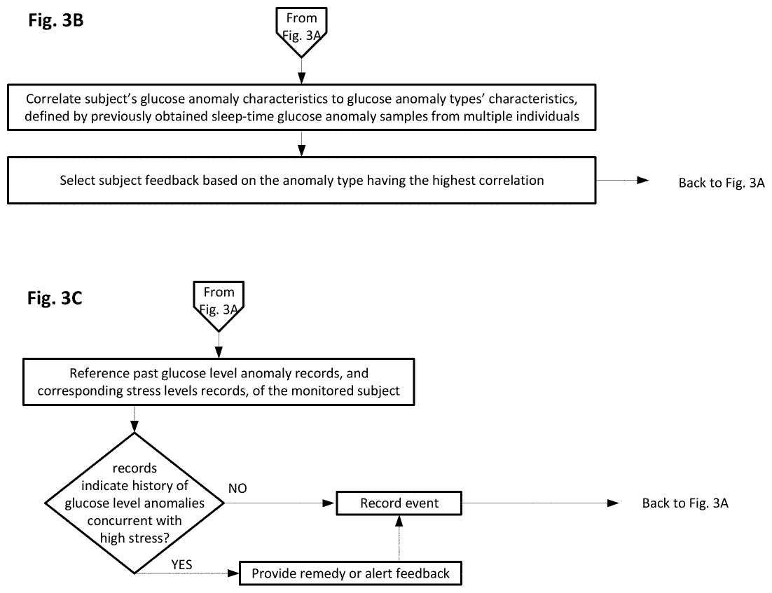

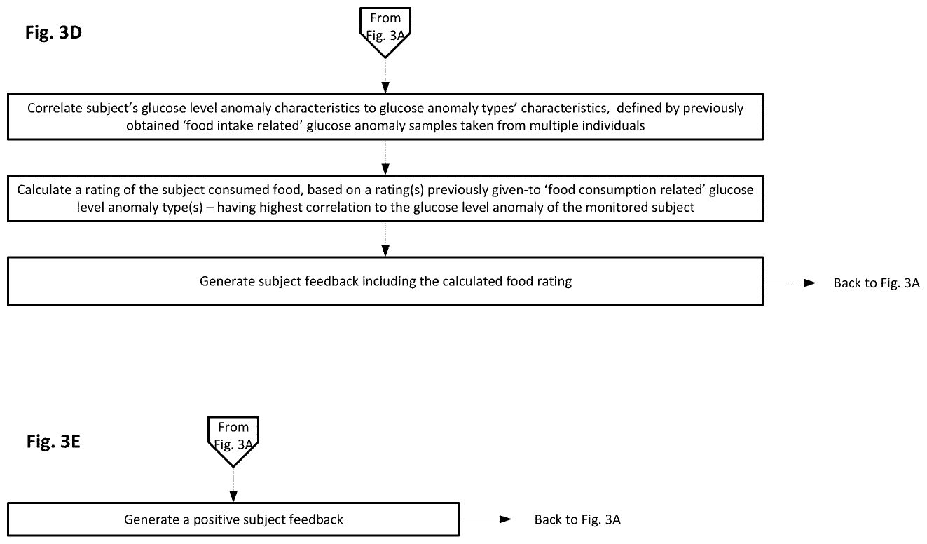

In FIG. 3A, there is shown a flowchart of the main steps executed as part of an exemplary glucose level anomaly analysis process, in accordance with some embodiments of the present invention;

In FIG. 3B, there is shown a flowchart of the main steps executed as part of an exemplary process executed upon detection of a sleep related glucose anomaly in FIG. 3A process, in accordance with some embodiments of the present invention;

In FIG. 3C, there is shown a flowchart of the main steps executed as part of an exemplary process executed upon detection of a stress level related glucose anomaly in FIG. 3A process, in accordance with some embodiments of the present invention;

In FIG. 3D, there is shown a flowchart of the main steps executed as part of an exemplary process executed upon detection of a food intake related glucose anomaly in FIG. 3A process, in accordance with some embodiments of the present invention;

In FIG. 3E, there is shown a flowchart of the main steps executed as part of an exemplary process executed upon detection of a physical activity related glucose anomaly in FIG. 3A process, in accordance with some embodiments of the present invention;

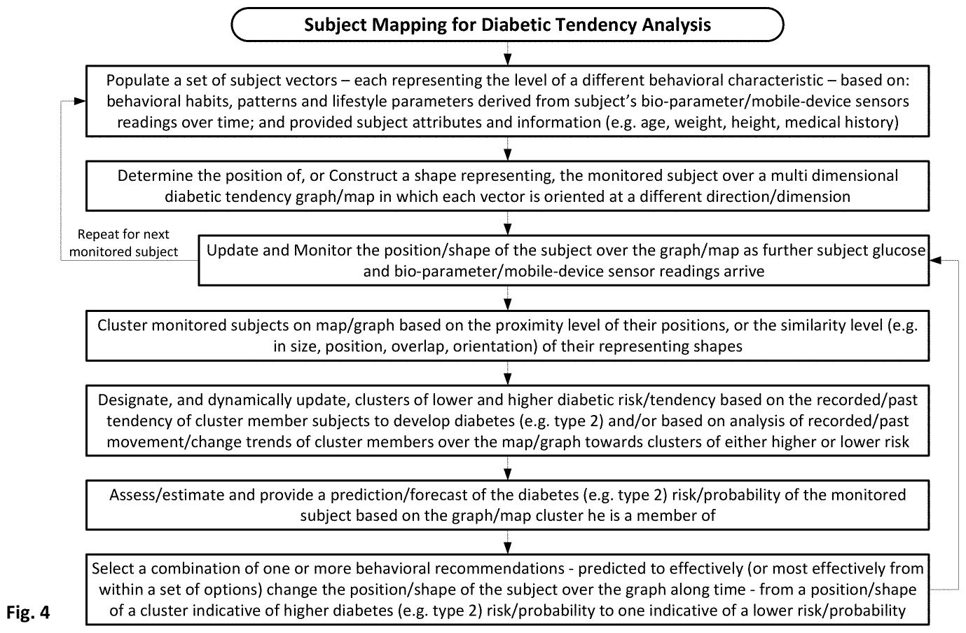

In FIG. 4, there is shown a flowchart of the main steps executed as part of an exemplary subject mapping for diabetic tendency analysis process, in accordance with some embodiments of the present invention;

In FIG. 5, there is shown an exemplary subject's glucose level sensor measured readings along time, a subject glucose level baseline/signature generated based on the readings and anomalies in the readings detected/selected by reference to the baseline, in accordance with some embodiments of the present invention;

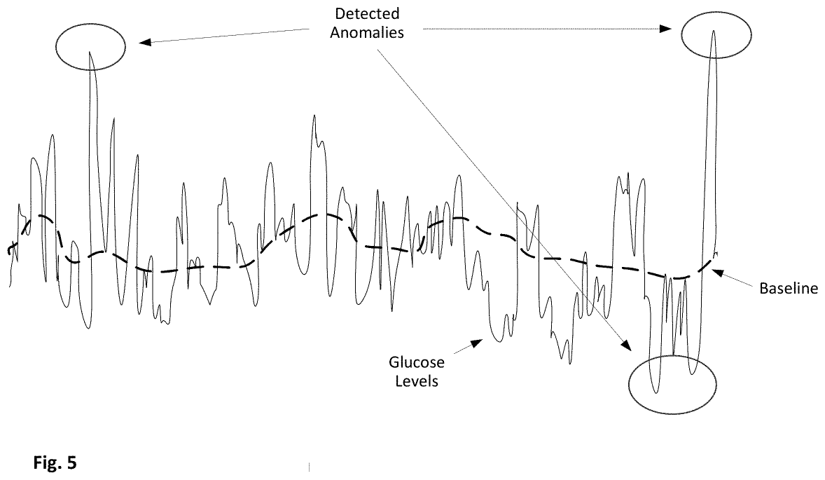

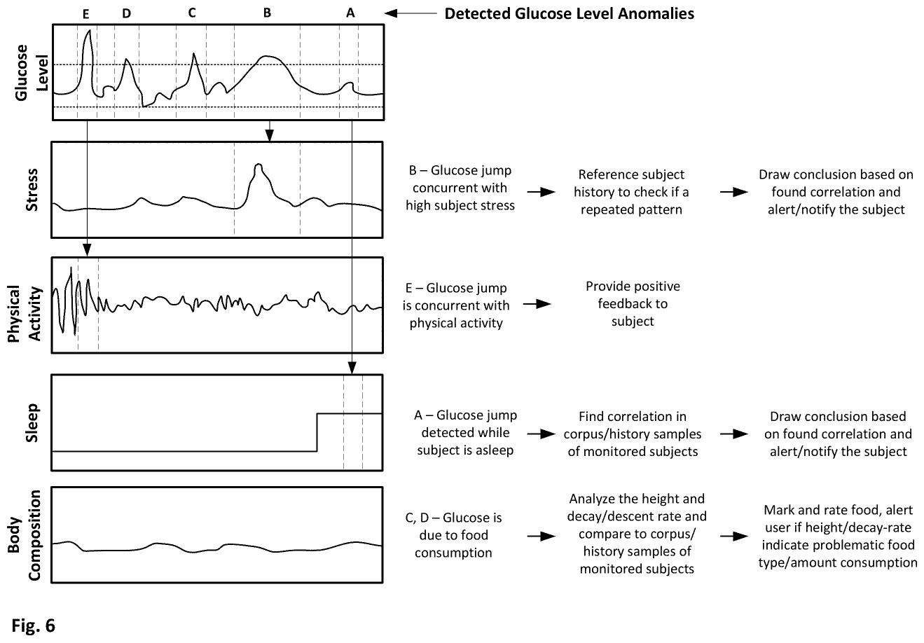

In FIG. 6, there is shown a diagram depicting the reference of different glucose level anomalies to time associated behavioral and physiological conditions of the monitored subject and the resulting conclusion and action/feedback in each case, in accordance with some embodiments of the present invention;

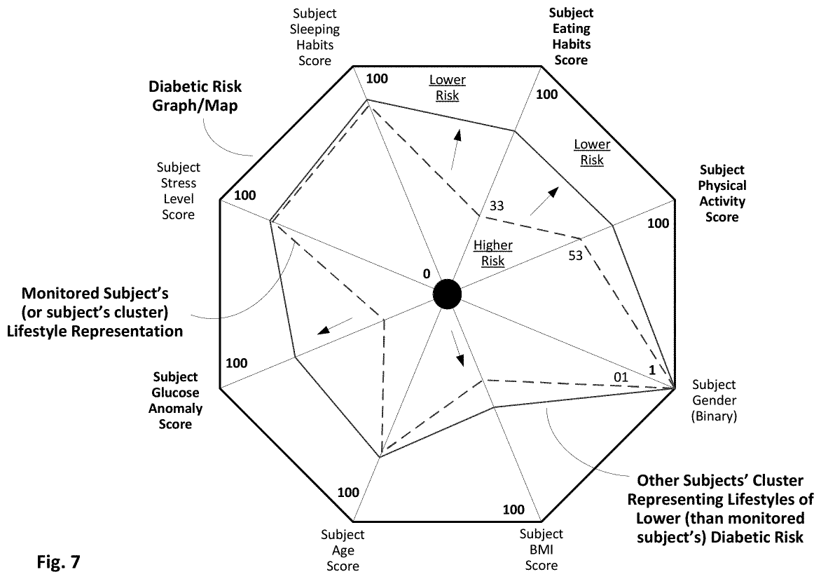

In FIG. 7, there is shown a schematic diagram depicting an exemplary diabetic risk map/graph, including subject scoring vectors, a monitored subject's lifestyle representation on the map/graph and other subjects' cluster representing lifestyles of lower diabetic risk, in accordance with some embodiments of the present invention;

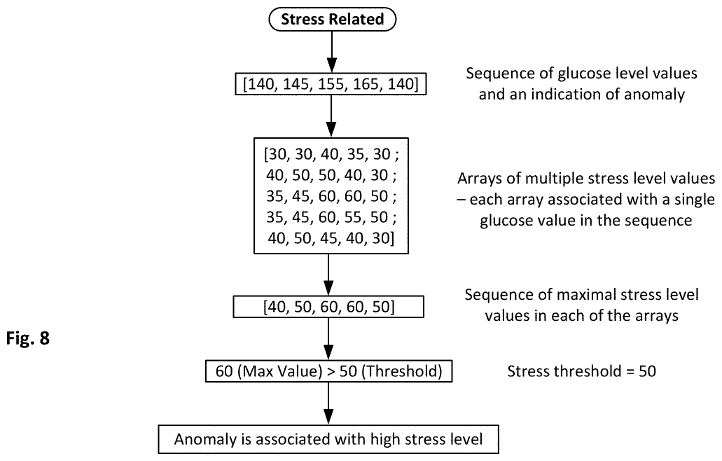

In FIG. 8, there is shown a flowchart of a schematic execution example of a decision process for estimating whether a glucose level anomaly is stress related, in accordance with some embodiments of the present invention;

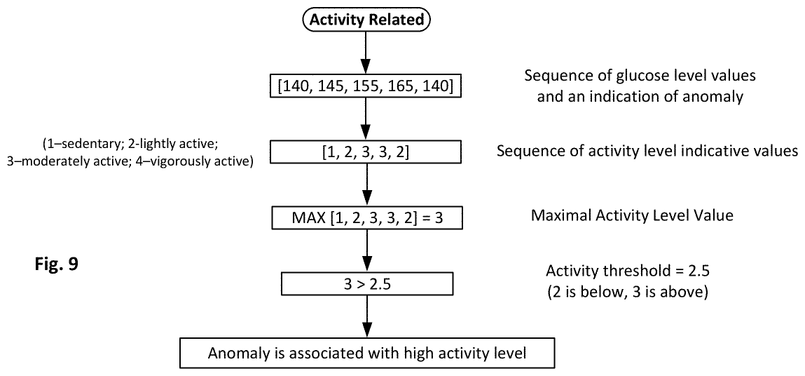

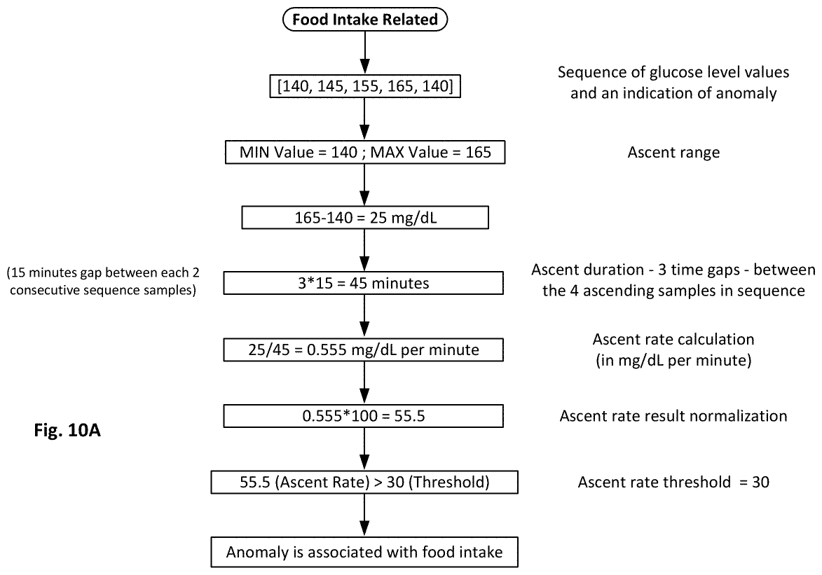

In FIG. 9, there is shown a flowchart of a schematic execution example of a decision process for estimating whether a glucose level anomaly is activity related, in accordance with some embodiments of the present invention;

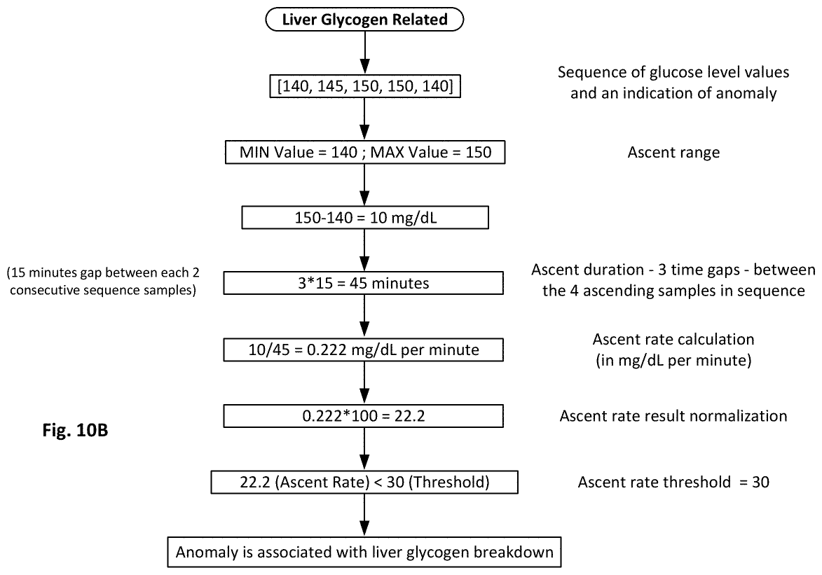

In FIG. 10A, there is shown a flowchart of a schematic execution example of a decision process for estimating whether a glucose level anomaly is food intake related, in accordance with some embodiments of the present invention;

In FIG. 10B, there is shown a flowchart of a schematic execution example of a decision process for estimating whether a glucose level anomaly is liver glycogen being broken related, in accordance with some embodiments of the present invention;

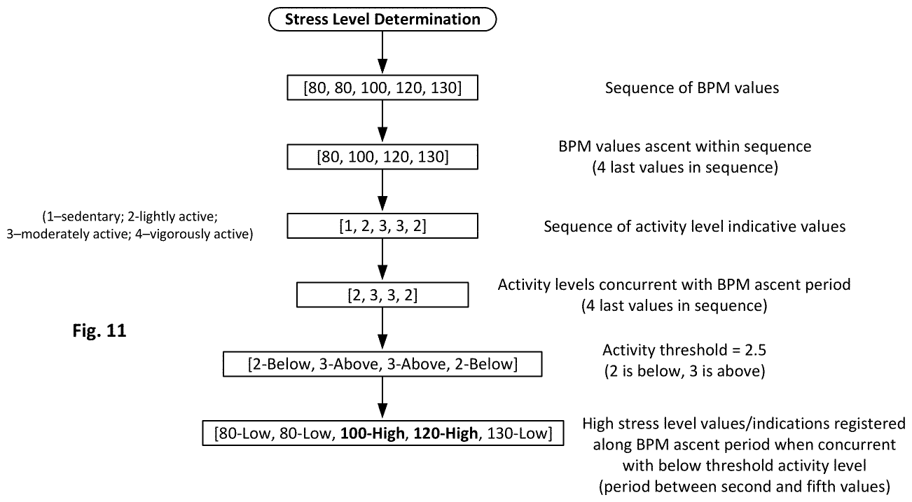

In FIG. 11, there is shown a flowchart of a schematic execution example of a decision process for estimating/determining the stress level of monitored subject, based on the subject's BPM and activity level, in accordance with some embodiments of the present invention;

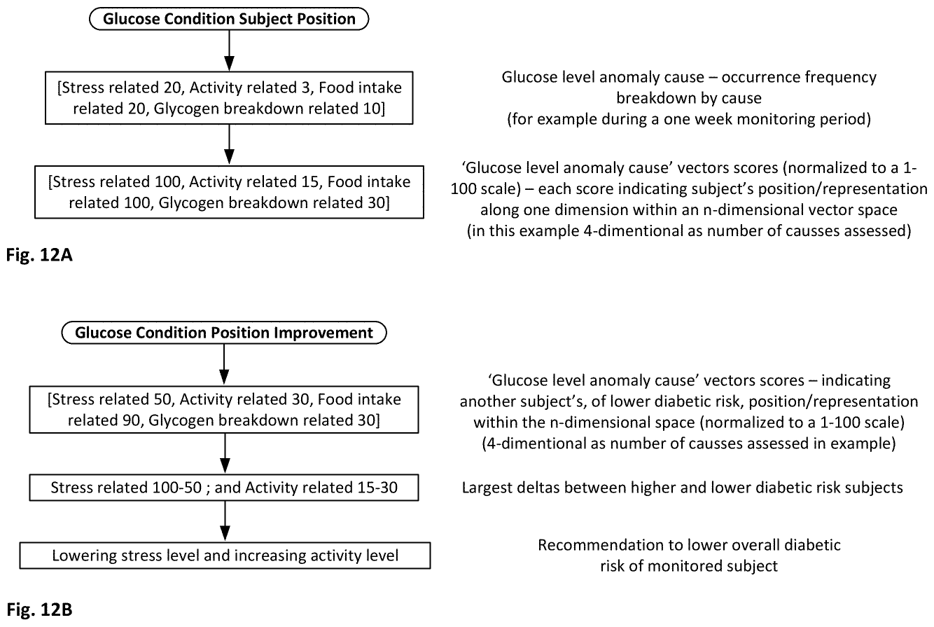

In FIG. 12A, there is shown a flowchart of a schematic execution example of a decision process for positioning/representing a monitored subject over an n-dimensional vector map/space, in accordance with some embodiments of the present invention;

In FIG. 12B, there is shown a flowchart of a schematic execution example of a decision process for generating/selecting a recommendation for lowering the diabetic risk of a monitored subject, based on the positioning of the monitored subject over an n-dimensional vector ‘diabetic risk map/space, in relation to the positioning of another, one or more, monitored subject who is positioned at a map/space region of lower risk, in accordance with some embodiments of the present invention;

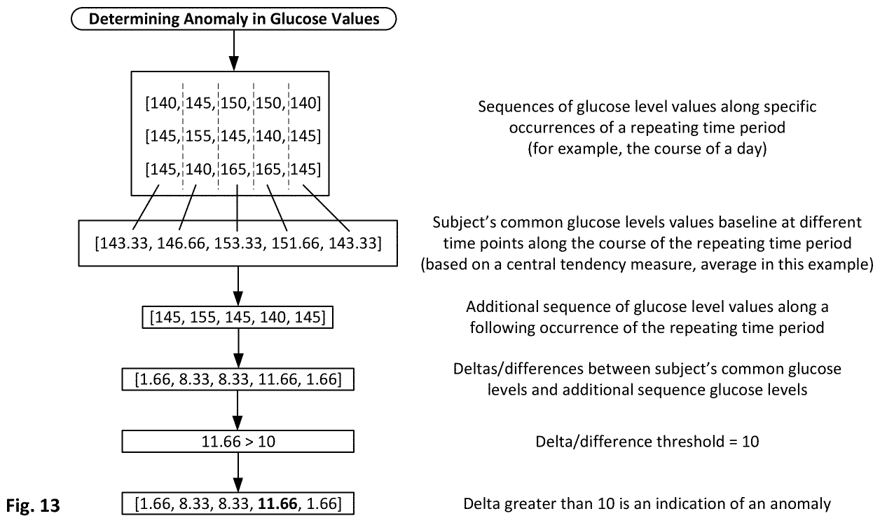

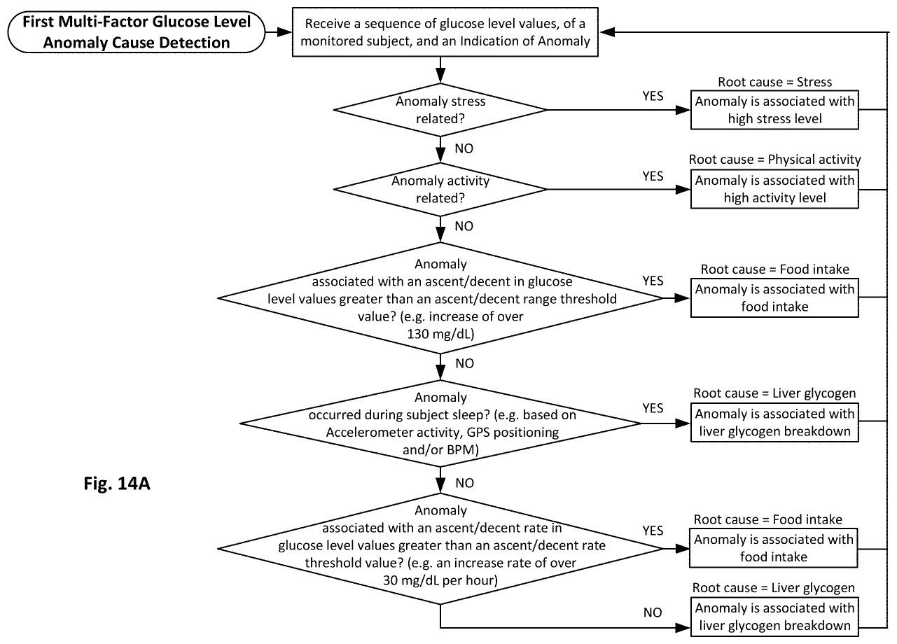

In FIG. 13, there is shown a flowchart of a schematic execution example, of a decision process for determining anomaly in glucose values, based on the generation of a monitored subject glucose level baseline and the comparison of following glucose level sequences of the monitored subject to the generated baseline, in accordance with some embodiments of the present invention;

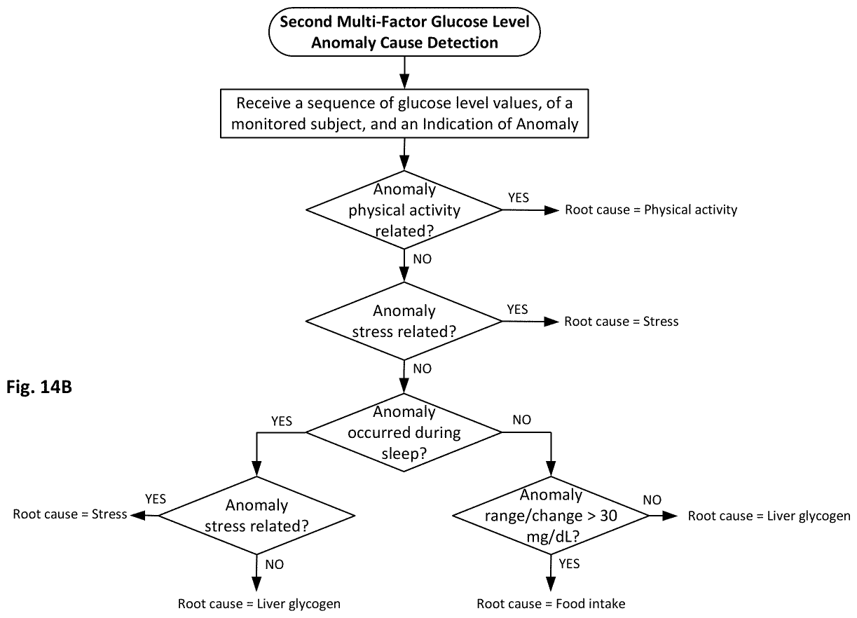

In FIG. 14A, there is shown a flowchart of the steps executed as part of an implementation of a first exemplary ‘multi-factor glucose level anomaly cause detection’ process, based on an integration/interrelation of multiple glucose level anomaly detection functions and processes, in accordance with some embodiments of the present invention; and

In FIG. 14B, there is shown a flowchart of the steps executed as part of an implementation of a second exemplary ‘multi-factor glucose level anomaly cause detection’ process, based on an integration/interrelation of multiple glucose level anomaly detection functions and processes, in accordance with some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals or element labeling may be repeated among the figures to indicate corresponding or analogous elements.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Unless specifically stated otherwise, as apparent from the following discussions, it is appreciated that throughout the specification discussions utilizing terms such as “processing”, “computing”, “calculating”, “determining”, or the like, may refer to the action and/or processes of a computer, computing system, computerized mobile device, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In addition, throughout the specification discussions utilizing terms such as “storing”, “hosting”, “caching”, “saving”, or the like, may refer to the action and/or processes of ‘writing’ and ‘keeping’ digital information on a computer or computing system, or similar electronic computing device, and may be interchangeably used. The term “plurality” may be used throughout the specification to describe two or more components, devices, elements, parameters and the like.

Some embodiments of the invention, may for example take the form of an entirely hardware embodiment, an entirely software embodiment, or an embodiment including both hardware and software elements. Some embodiments may be implemented in software, which includes but is not limited to, any combination of: firmware, resident software, microcode, or the like. Some embodiments may be implemented in hardware, which includes but is not limited to, any combination of: a processor, memory and data storage components, a power source, communication circuitry, I/O interfaces, cards and devices, programmable arrays, systems on chip, or the like. Some embodiments may be implemented using a combination of hardware and software, which includes but is not limited to, any combination of the above hardware and software types and components.

Furthermore, some embodiments of the invention may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device, for example a computerized device running a web-browser.

In some embodiments, the medium may be an electronic, magnetic, optical, electromagnetic, infrared, or semiconductor system (or apparatus or device) or a propagation medium. Some demonstrative examples of a computer-readable medium may include a semiconductor or solid state memory, magnetic tape, a removable computer diskette, a random access memory (RAM), a read-only memory (ROM), a rigid magnetic disk, and an optical disk. Some demonstrative examples of optical disks include compact disk-read only memory (CD-ROM), compact disk-read/write (CD-R/W), and DVD.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution. The memory elements may, for example, at least partially include memory/registration elements on the user device itself.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Functions, operations, components and/or features described herein with reference to one or more embodiments, may be combined with, or may be utilized in combination with, one or more other functions, operations, components and/or features described herein with reference to one or more other embodiments.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “includes”, “including”, “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention, and is not intended to limit the invention to the specific embodiments illustrated by the figures or description below.

Embodiments of the present invention include a glucose monitoring, analysis and remedy system, including a glucose analysis sever/logic for generating a monitored subject glucose level baseline, also referred to herein as a ‘Personal Glucose Signature’ or PGS—representing the subject's common/usual glucose level values along the course of a day.

The subject glucose level baseline may be calculated based on median values, and/or other measure(s) of central tendency, of monitored subject glucose level readings, collected by a non-invasive sensor assembly and communicated through a monitored subject mobile device application, along with mobile device sensors data.

Newly received, monitored subject glucose level readings sets may be compared to subject glucose level baseline values sets relating to the same, or substantially the same, time of the day, to detect anomalies between the two value sets which are indicative of specifically characterized glucose level jumps/rises.

An indication of a glucose level anomaly, may be analyzed by reference of one or more subject behavioral or physiological conditions concurrent with the anomaly, wherein the behavioral or physiological conditions are derived/calculated/concluded based on readings/data from a combination of one or more of the assembly sensors and the mobile device sensors.

A monitored subject feedback, and/or an alert/notification, may be generated or selected at least partially based on the type, level and/or characteristics of the behavioral or physiological condition experienced by the monitored subject concurrently with the glucose level anomaly.

The behavioral or physiological conditions may, in accordance with some embodiments, include a combination of at least the following conditions and may be non-invasively and continuously/intermittently sensed/derived/calculated/concluded as follows:
Glucose Level—Using a Photoplethysmograph (PPG) sensor, Bioimpedance sensor, or any other glucose sensor or sensing technique.
Sleep—Based on a combination of an accelerometer (on sensor-composite, smart-watch, mobile device) readings and monitored subject heartbeat rate sensor readings.
Stress Level—Based on measured glucose level anomalies and disorders, Galvanic Skin Response (GSR) sensor, or other.
Body Mass Index (BMI)—Based on Bioimpedance sensor readings.
Physical Activity—Based on an accelerometer (on sensor-composite, smart-watch, mobile device) readings. May also be indicative on length, frequency, intensity, and type (e.g. Aerobic/Cardio, Anaerobic) and may be assisted by a GPS or other positioning sensor/technique.
Liver Glycogen—Deducted based on the detection (as described above) of a monitored subject which is: a sleep and thus not eating, not shown to be experiencing stress and has concurrently experienced a Glucose level rise/anomaly. Accordingly—if no food intake, stress, or physical activity is found to be associated with the detected glucose rise/anomaly—the remaining, deducted, glucose level effecting condition, is liver or cell glycogen being broken and glucose being released to bloodstream.
   Food Intake—analysis of glucose level anomalies behavior patterns—for example by using anomaly pattern data as training data for an AI/NN learning process, to later cluster/group similarly patterned anomalies—may be utilized for out-filtering of conditions not relevant, (e.g. not having the same/similar pattern, to a detected glucose anomaly. Accordingly, a given anomaly may be regarded as: non-activity, non-stress and non-liver-related—and hence, concluded to be food intake related.

Monitored subject assembly/mobile-device sensors data and subject behavioral or physiological condition derived therefrom, along with other provided subject specific information, may be analyzed over time to generate multiple subject vectors, each representing a subject's lifestyle (i.e. measured along time) score associated with another condition.

Multiple condition vectors scores of a given subject may be used to find/construct a position/shape representing the subject over a multi-condition diabetic risk graph/map, a general subject diabetic risk/tendency score may be generated based on the conditions scores.

Graph/map representations of multiple subjects' may be clustered, wherein graph/map representations, substantially similarly positioned/shaped over the graph/map, may be associated with a same cluster.

Clusters of higher and lower diabetic risk may be designated based on their associated subjects' general risk/tendency levels. The diabetic risk of a given subject may be predicted/forecasted based on the prior recorded movements, of former subject's-cluster members, towards/in-the-direction of either higher or lower risk/tendency clusters over the graph/map.

One or more specifically selected/constructed lifestyle recommendations, to efficiently/rapidly—while optionally also factoring subject recommendation selection/preference—mobilize/change, over time, the subject's graph/map representation towards/to a graph/map representation associated with a subjects' cluster of a lower diabetic risk score than the current monitored-subject, or monitored-subject cluster, risk score.

The Glucose Monitoring, Analysis, and Remedy Suggestion System, according to some embodiments of the present invention, may comprise a combination of at least the following described components.

(I) An exemplary non-invasive sensors set/assembly/composite, in accordance with some embodiments, including:

(1) Two or more bio-parameter sensors including at least a glucose level indicative sensor(s) and, a combination of a stress level indicative sensor(s), food intake indicative sensor(s), physical activity/exercise indicative sensor(s) and/or a sleeping state indicative sensor(s), to collect/acquire samples from a monitored subject and/or the subject's environment.

(2) Sensors driver/interface circuitry.

(3) Sensors signals/data processing circuitry.

(4) Communication circuitry.

(5) A sensors set/assembly/composite controller/control-logic.

And/or (6) Battery and power/recharge circuits.

An exemplary non-invasive sensors set/assembly/composite, in accordance with some embodiments, may take the form of any smart watch or other wearable device adapted to measure monitored/wearing subject's bio-parameters and/or other subject related parameters and communicate them to a mobile device or remote/networked computing device. A system, in accordance with some embodiments, may analyze and provide feedback as described herein, based on data provided from a third party sensors set/assembly/composite, smart watch, or digital/online wearable.

(II) An exemplary Mobile device application, in accordance with some embodiments, including:

(1) A device communication circuitry/drivers interface to:

(1.1) Receive digital data values/streams/readings representing sensors signal samplings from the monitored subject's sensors set/assembly/composite; Relay the received digital data values/streams to a glucose analysis block/server/cloud; and Receive monitored subject notifications/reports from the glucose analysis block/server/cloud.

(2) A device input/output circuitry/drivers interface to:

(2.1) Present the received monitored subject notifications/reports through output components of the mobile device (e.g. screen, speakers, tactile); and/or Receive monitored subject inputs—including subject behavioral information, commands and inquiries—through input components of the mobile device (e.g. keyboard, microphone, camera).

(3) A device sensors circuitry/drivers interface to:

(3.1) Receive digital data values/streams/readings representing sensors signal samplings from the monitored subject's mobile device (e.g. GPS, accelerometer); and/or Provide the received data to the communication circuitry/drivers interface for relay to the glucose analysis block/server/cloud.

(III) A glucose analysis block/server/cloud/logic, including:

(1) A glucose level anomaly detection module for: (1.1) Receiving a first sequence of multiple glucose level samples of a monitored subject; Generating a monitored subject glucose level baseline, or PGS—representing the subject's common sugar levels values along the course of a day; Receiving a second sequence of multiple glucose level samples of the monitored subject; Comparing one or more of the samples from the second sequence to the generated subject glucose level baseline; and Determining an anomaly in the monitored subject's glucose level upon comparison results indicating a difference (e.g. difference in: size, frequency, change rate, lasting period, or any combination thereof)—beyond a predetermined threshold level—between the monitored subject's second sequence samples values and the generated baseline values for the same period of the day.

According to some embodiments, (1.1.1) values of at least some of the one or more samples from the second sequence may be used to update the monitored subject's glucose level baseline.

According to some embodiments, (1.1.2) multiple detected anomalies in the monitored subject's glucose levels may be grouped based on the magnitude/scale/size of difference between each anomaly's indicating samples (from the second sequence) and the subject's glucose level baseline.

According to some embodiments, (1.1.2.1) the detected anomalies groups may include at least a ‘minor-difference’, a ‘medium-difference’ and a ‘high-difference’ group.

According to some embodiments, (1.1.3) specific difference magnitude/scale/size and/or scenarios/sets/deltas, between samples from the second sequence and the generated subject glucose level baseline, may be defined and pre-associated with corresponding glucose level change causes; Wherein upon detection of a specific difference magnitude/scenario between samples from the second sequence to the generated subject glucose level baseline, the corresponding pre-associated glucose level change cause may be communicated to the monitored subject or to a medical consultant.

(2) A glucose level anomaly analysis module for: (2.1) Determining/Receiving-Indication-of an anomaly in a monitored subject's glucose level; Referencing values sampled, during, or in proximity to, the time period of the glucose level anomaly, by the one or more bio-parameter sensors monitoring the subject and/or by one or more subject mobile device sensors; Detecting, based on values sampled by one (or by a combination) of the bio-parameter/mobile-device sensors, a physiological or behavioral condition of the monitored subject that occurred during, or in proximity to, the time period of the glucose level anomaly; and Selecting/Generating a monitored subject feedback (e.g. alert, remedy, behavioral-recommendation) at least partially based on the detected physiological or behavioral condition of the subject during the time period of the glucose level anomaly.

According to some embodiments, (2.2) the bio-parameter/mobile-device sensors may provide sensors data indicative, or enabling the derivation/calculation/conclusion as described herein, of the monitored subject's: stress level, food intake status, physical activity/exercise status and/or whether subject is a sleep—at a given time point and/or during a given time period.

According to some embodiments, (2.2.1) an indication that the monitored subject was asleep during a detected anomaly in the monitored subject's glucose level, may trigger the correlation of the subject's glucose level anomaly characteristics to one or more glucose level anomaly types' characteristics, defined by previously obtained sleep-time glucose anomaly samples taken from multiple individuals; wherein a monitored subject feedback (e.g. alert, remedy, behavioral-recommendation) is selected at least partially based on the anomaly type having the highest correlation to the detected sleep-time glucose level anomaly of the monitored subject.

According to some embodiments, (2.2.2) an indication that the monitored subject was experiencing high stress levels during a detected anomaly in the monitored subject's glucose level, may trigger the reference of past glucose level anomaly records, and their time corresponding stress levels records, of the monitored subject; wherein a first monitored subject feedback (e.g. alert, remedy, behavioral-recommendation) is selected if the referenced subject records indicate a history of repeating glucose level anomalies occurring concurrently with high stress levels and, a second monitored subject feedback (e.g. alert, remedy, behavioral-recommendation) is selected if the referenced subject records indicate no history of repeating glucose level anomalies occurring concurrently with high stress levels.

According to some embodiments, (2.2.3) an indication that the detected anomaly in the monitored subject's glucose level is associated with food intake, may trigger the correlation of the subject's glucose level anomaly characteristics (e.g. height, decay) to one or more glucose level anomaly types' characteristics, defined by previously obtained ‘food intake related’ glucose anomaly samples taken from multiple individuals; wherein a monitored subject feedback (e.g. alert, remedy, behavioral-recommendation) may include a rating of the subject consumed food, the rating calculated at least partially based on a rating previously given-to/associated-with the specific ‘food intake related’ glucose level anomaly type—found to have the highest correlation to the detected ‘food intake related’ glucose level anomaly of the monitored subject.

According to some embodiments, (2.2.4) an indication that the detected anomaly in the monitored subject's glucose level is associated with physical activity/exercise, may trigger a positive monitored subject feedback (e.g. encouragement, related benefits, additional positive behavior recommendations [e.g. physical activity, nutrition, stress relief]).

(3) A monitored subject glucose condition mapping (positioning, monitoring, recommending and updating) module for: (3.1) Populating—for a monitored subject—multiple, dynamic, characteristic vectors, with respective scores, wherein the score for each subject vector, may be based on: subject provided information/values, sensor-samples indicated behavioral and physiological conditions/characteristics/values patterns/schemes of the subject showing over a monitoring period, and/or glucose anomaly conditions/characteristics/values patterns/schemes of the subject showing over a monitoring period (e.g. anomaly causes'-frequencies, amplitudes, durations, and ascent/decent rates); and Determining/calculating the positioning/representative-shape of the subject on a multi-dimension glucose condition/risk graph/map, based on multi-characteristic vectors values.

According to some embodiments, glucose condition mapping may further include (3.2) Intermittently updating the multi-characteristic vectors, based on additional sensor samples data received for the subject, and thus the determined positioning/representation of the subject on the graph/map.

According to some embodiments, glucose condition mapping may further include (3.2.1) Generating a monitored subject feedback including an optimized behavioral-recommendation(s) suggesting specific behavioral/lifestyle changes, steps, steps scope, or steps combinations—to most efficiently (minimal steps/resources) or rapidly improve the positioning/representation of the subject on the graph/map to a positioning/representation representing a lower monitored subject diabetic risk/tendency.

According to some embodiments, glucose condition mapping may further include (3.2.2) Generating a monitored subject feedback including an optimized behavioral-recommendation(s) suggesting specific behavioral/lifestyle changes, steps, steps scope, or steps combinations—to most effectively prevent the moving of the positioning/representation of the subject on the graph/map towards, a position(s) associated with a higher probability for a glucose levels health condition such as type 2 diabetes.

(4) A monitored subject feedback and scoring module to: Integrate subject feedbacks (e.g. physiological and behavioral condition ranks, scores, alerts, remedies, behavioral-recommendations) from the detection, analysis and mapping modules described herein—optionally in combination with feedback/filtering/tuning/curating information/instructions provided by a human medical consultant to which sensor samples derived data was presented into a monitored subject notification/report/feedback.

And/or (5) Communication circuitry and components/modules to: Receive digital data values/streams/readings representing sensors signal samplings from monitored subjects' mobile device applications; and Communicate monitored subject notifications/scores/recommendations/reports to respective subjects' mobile device applications.

In FIG. 1A, there is shown a block diagram of an exemplary system for glucose monitoring, analysis, and remedy, including components thereof and interconnections there between, in accordance with some embodiments of the present invention.

The system shown includes:

(I) A Non-invasive sensors set/assembly/composite, monitoring a subject, including: (1) Two or more bio-parameter sensors including at least a glucose level indicative sensor(s) and, a combination of a stress level indicative sensor(s), food intake indicative sensor(s), physical activity/exercise indicative sensor(s) and/or a sleeping state indicative sensor(s), to collect/acquire samples from a monitored subject and/or the subject's environment; (2) Sensors driver/interface circuitry; (3) Sensors signals/data processing circuitry; (4) Communication circuitry; (5) A sensors set/assembly/composite controller/control-logic; And (6) Battery and power/recharge circuits.

(II) A Mobile device application, including: (1) A device communication circuitry/drivers interface; (2) A device input/output circuitry/drivers interface; and (3) A device sensors circuitry/drivers interface;

(III) A glucose analysis block/server/cloud/logic, including: (1) A glucose level anomaly detection module comprising a subject data normalizer & baseline generator and an anomaly detector; (2) A glucose level anomaly analysis module comprising a reference condition determination logic, an anomaly to reference condition matching logic and an anomaly cause estimation logic; (3) A monitored subject glucose condition mapping (positioning, monitoring, recommending and updating) module comprising a behavioral characteristic vectors population logic, a position determination and clustering logic and a position monitoring updating & forecasting logic, wherein the position determination and position monitoring logics are functionally connected to a rule-set/ML/DL/AI/NN based decision model/machine/logic; (4) A monitored subject feedback, recommendation and scoring module; (5) Communication circuitry and components/modules.

In FIG. 1B, there is shown a flowchart of the main steps executed as part of an exemplary glucose monitoring, analysis, and remedy process, in accordance with some embodiments of the present invention.

Shown steps include: (1) Monitor a subject's glucose level readings and bio-parameter/mobile-device sensor readings; (2) Detect anomalies in monitored subject's glucose level; (3) Estimate cause of anomalies in monitored subject's glucose level and provide matching feedback; (4) Determine and monitor subject's general glucose/diabetic condition and provide risks, remedies and progress feedbacks.

In FIG. 2, there is shown a flowchart of the main steps executed as part of an exemplary glucose level anomaly detection process, in accordance with some embodiments of the present invention.

Shown steps include: (1) Receive a monitored subject's glucose level readings and bio-parameter/mobile-device sensor readings; (2) if Sufficient amount of glucose level readings accumulated continue, else go to (1); (3) Generate a monitored subject's glucose level baseline based on glucose readings received over a time period; (4) Receive further monitored subject's glucose level readings and bio-parameter/mobile-device sensor readings; (5) Detect anomalies between the subject's baseline and newly received glucose level readings; and (6) Update baseline based on newly received readings and return to (4).

In FIG. 3A, there is shown a flowchart of the main steps executed as part of an exemplary glucose level anomaly analysis process, in accordance with some embodiments of the present invention.

Shown steps include: (1) Receive an indication of an anomaly in a monitored subject's glucose level; (2) Reference values sampled, during, or in proximity to, the time period of the glucose level anomaly, by the one or more bio-parameter/mobile-device sensors monitoring the subject; (3) Detect, based on values sampled by one (or by a combination) of the bio-parameter/mobile device sensors, a physiological or behavioral condition of the monitored subject during, or in proximity to, the time period of the glucose level anomaly; (4) Select a monitored subject feedback (e.g. alert, remedy, behavioral-recommendation) at least partially based on the detected physiological or behavioral condition of the subject during the time period of the glucose level anomaly, wherein: (a) if the subject was asleep go to FIG. 3B process, else continue; (b) if the subject experienced high stress levels go to FIG. 3C process, else continue; (c) if the subject consumed food go to FIG. 3D process, else continue; (d) if the subject performed physical activity go to FIG. 3E process, else continue; and (e) if other physiological or behavioral condition detected provide feedback in accordance with condition result, optionally factoring other condition(s) results, if not, provide feedback indicating subject glucose anomaly caused by liver/cells dissolved glycogen and return to (1).

In FIG. 3B, there is shown a flowchart of the main steps executed as part of an exemplary process executed upon detection of a sleep related glucose anomaly in FIG. 3A process, in accordance with some embodiments of the present invention.

Shown steps include: (1) Correlate subject's glucose anomaly characteristics to glucose anomaly types' characteristics, defined by previously obtained sleep-time glucose anomaly samples from multiple individuals; and (2) Select subject feedback based on the anomaly type having the highest correlation and return to start of FIG. 3A process.

In FIG. 3C, there is shown a flowchart of the main steps executed as part of an exemplary process executed upon detection of a stress level related glucose anomaly in FIG. 3A process, in accordance with some embodiments of the present invention.

Shown steps include: (1) Reference past glucose level anomaly records, and corresponding stress levels records, of the monitored subject; (2) if records indicate history of glucose level anomalies concurrent with high stress, then provide remedy or alert feedback and record event, else record event—then return to start of FIG. 3A process.

In FIG. 3D, there is shown a flowchart of the main steps executed as part of an exemplary process executed upon detection of a food intake related glucose anomaly in FIG. 3A process, in accordance with some embodiments of the present invention.

Shown steps include: (1) Correlate subject's glucose level anomaly characteristics to glucose anomaly types' characteristics, defined by previously obtained ‘food intake related’ glucose anomaly samples taken from multiple individuals; (2) Calculate a rating of the subject consumed food, based on a rating(s) previously given-to ‘food consumption related’ glucose level anomaly type(s)—having highest correlation to the glucose level anomaly of the monitored subject; (3) Generate subject feedback including the calculated food rating and return to start of FIG. 3A process.

In FIG. 3E, there is shown a flowchart of the main steps executed as part of an exemplary process executed upon detection of a physical activity related glucose anomaly in FIG. 3A process, in accordance with some embodiments of the present invention.

Shown step includes: (1) Generate a positive subject feedback and return to start of FIG. 3A process.

In FIG. 4, there is shown a flowchart of the main steps executed as part of an exemplary subject mapping for diabetic tendency analysis process, in accordance with some embodiments of the present invention.

Shown steps include: (1) Populate a set of subject vectors—each representing the level of a different behavioral characteristic—based on: behavioral habits, patterns and lifestyle parameters derived from subject's bio-parameter/mobile-device sensors readings over time; and provided subject attributes and information (e.g. age, weight, height, medical history); (2) Determine the position of, or Construct a shape representing, the monitored subject over a multi-dimensional diabetic tendency graph/map in which each vector is oriented at a different direction/dimension; (3) Update and Monitor the position/shape of the subject over the graph/map as further subject glucose and bio-parameter/mobile-device sensor readings arrive, repeating steps (1)-(3) for the next monitored subject; (4) Cluster monitored subjects on map/graph based on the proximity level of their positions, or the similarity level (e.g. in size, position, overlap, orientation) of their representing shapes; (5) Designate, and dynamically update, clusters of lower and higher diabetic risk/tendency based on the recorded/past tendency of cluster member subjects to develop diabetes (e.g. type 2) and/or based on analysis of recorded/past movement/change trends of cluster members over the map/graph towards clusters of either higher or lower risk; (6) Assess/estimate and provide a prediction/forecast of the diabetes (e.g. type 2) risk/probability of the monitored subject based on the graph/map cluster he is a member of; (7) Select a combination of one or more behavioral recommendations—predicted to effectively (or most effectively from within a set of options) change the position/shape of the subject over the graph along time—from a position/shape of a cluster indicative of higher diabetes (e.g. type 2) risk/probability to one indicative of a lower risk/probability and return to step (3).

In FIG. 5, there is shown an exemplary subject's glucose level sensor measured readings along time, a subject glucose level baseline/signature generated based on the readings and anomalies in the readings detected/selected by reference to the baseline, in accordance with some embodiments of the present invention. Subject Glucose level anomalies detected by comparison to the generated subject baseline are circled in the figure. Other potential anomalies have been filtered out, as they are regarded as a non-anomaly when compared/assessed-in-relation-to the monitored subject baseline.

In FIG. 6, there is shown a diagram depicting the reference of different glucose level anomalies to time associated behavioral and physiological conditions of the monitored subject and the resulting conclusion and action/feedback in each case, in accordance with some embodiments of the present invention.

In the figure, the glucose levels graph of a monitored subject is shown along graphs of other, time corresponding, levels physiological/behavioral parameters of the subject. The first anomaly detected in the glucose values is (A)—a glucose jump detected while subject is asleep; accordingly, correlation in corpus/history samples of monitored subjects is found and conclusion based on found correlation drawn and alerted/notified to the subject. The second anomaly detected in the glucose values is (B)—a glucose jump concurrent with high subject stress; accordingly, reference subject history to check if a repeated pattern, draw conclusion based on found correlation and alert/notify the subject. The third and fourth anomalies detected in the glucose values are (C) and (D)—glucose jumps due to food consumption; accordingly, analyze the height and decay/descent rate, compare to corpus/history samples of monitored subjects, mark and rate food and alert user if height/decay-rate indicate problematic food type/amount consumption. The fifth anomaly detected in the glucose values is (E)—a glucose jump concurrent with physical activity; accordingly, provide positive feedback to subject.

In FIG. 7, there is shown a schematic diagram depicting an exemplary diabetic risk map/graph, including subject scoring vectors, a monitored subject's lifestyle representation on the map/graph and other subjects' cluster representing lifestyles of lower diabetic risk, in accordance with some embodiments of the present invention.

In the figure, there is shown an exemplary diabetic risk graph/map (shown octagon) including scoring vectors for each of multiple monitored-subject, and monitored-subject lifestyle, aspects/facets/characteristics. A monitored subject score, based on subject provided parameters and system sensors subject data collected and analyzed over time, is calculated/allocated for each of the graph/map vectors on a scale between 1-100 (or, 1 OR 0 for subject gender vector). The scores of the subject collectively define a shape (dotted lines) representing the monitored subject, and a monitored subject diabetic risk/tendency score is calculated based thereof.

A graph/map shape representing a cluster of other monitored subjects whose scores indicate lifestyles of lower (than monitored subject's) diabetic risk/tendency, is shown in full (non-dotted) lines. Monitored subject feedback, may be constructed to include specific lifestyle suggestions/recommendations directed to specific monitored subject lifestyle aspects/facets/characteristics scores, in order to effectively change, along time, the shape representation of the monitored subject to that of the lower diabetic risk/tendency cluster.

In the example of the figure, recommendations for better eating habits and physical activity habits may be suggested, to improve corresponding monitored subject scores that are currently low (33 and 53 respectively)—‘opening’ the shape representation of the subject on the graph/map in the directions of the two top arrows. Improvement in these scores may lead, over time, to better BMI and Glucose anomaly scores—further ‘opening’ the shape representation of the subject on the graph/map in the directions of the two bottom arrows.

According to some embodiments, scales of the scoring vectors for each of the multiple monitored-subject, and monitored-subject lifestyle, aspects/facets/characteristics—may be normalized, as exemplified in the figure, to a 1-100 and 0 OR 1 (Binary—subject gender) scales.

According to some embodiments, scoring vectors may relate to monitored subject affectable/changeable parameters, such as lifestyle aspects/facets/characteristics scores, while other diabetic risk components factored, are either unchangeable (e.g. gender), change uncontrollably (e.g. age) and/or are affected by other factored scores (e.g. BMI and Glucose anomalies scores—by physical activity and food intake habits).

According to some embodiments, different scoring vectors scores may be assigned different weights—representing the level of their contribution/affect to the general monitored subject diabetic risk/tendency score. For example, factors such as eating habits score, physical activity score and subject glucose anomaly score, may have more influence on the general subject score than factors such as sleeping habit scores.

According to some embodiments, a rule-set, a machine learning, an artificial intelligence (AI) and/or a neural network based decision model—may be utilized for: monitored-subjects graph/map representations clustering; for subjects and clusters representations comparisons; and/or for the forecasting of a monitored subject's graph/map representation change/movement in the future, towards either a higher diabetic risk or a lower diabetic risk representation—wherein the AI/NN training data may at least partially include past subjects' graph/map representation changes/movements data, of monitored subjects representation changes/movements initiating from the a representation substantially similar to the current monitored subject representation.

According to some embodiments, a system for glucose level anomaly cause detection may comprise: (1) one or more processors; (2) a communication module functionally associated with the one or more processors and adapted for: (a) receiving a sequence of glucose level values of a monitored subject and an Indication of a glucose levels anomaly within the received values sequence; and (b) receiving a sequence of stress level values, of the monitored subject, sampled concurrently with the sequence of glucose level values; and (3) a memory functionally associated with, and adapted for storing instructions for execution by, the one or more processors, for: (a) comparing the received stress level values to a predetermined stress level threshold; and (b) indicating that the glucose level anomaly is associated with a high stress level of the monitored subject if the received stress level values surpass the predetermined stress level threshold.

In FIG. 8, there is shown a flowchart of a schematic execution example of a decision process for estimating whether a glucose level anomaly is stress related, in accordance with some embodiments.

In the FIG. 8 example, the following execution steps are exemplified: (1) receiving of a sequence of glucose level values and an indication of anomaly; (2) receiving arrays/vectors of multiple stress level values—each array/vector, time associated with a single glucose value in the sequence; (3) extracted sequence of maximal stress level values in each of the arrays/vectors; (4) comparing the maximal value in the extracted sequence to a stress level threshold; and (5) indicating that the glucose anomaly is associated with high stress level, as the maximal stress value surpassed the stress level threshold.

According to some embodiments, the communication module may be adapted for: (a) receiving a sequence of activity level values, of the monitored subject, sampled concurrently with the sequence of glucose level values; and the memory may be adapted for storing instructions, for execution by the one or more processors, for: (a) comparing the received activity level values to a predetermined activity level threshold; and, (b1) indicating that the glucose level anomaly is associated with a high activity level of the monitored subject if the received activity level values surpass the predetermined activity level threshold, or (b2) indicating that the glucose level anomaly is associated with a high activity level of the monitored subject if both, the received stress level values remain under the predetermined stress level threshold and the received activity level values surpass the predetermined activity level threshold.

In FIG. 9, there is shown a flowchart of a schematic execution example of a decision process for estimating whether a glucose level anomaly is activity related, in accordance with some embodiments.

In the FIG. 9 example, the following execution steps are exemplified: (1) receiving of a sequence of glucose level values and an indication of anomaly; (2) receiving a sequence of activity level indicative values—each value, time associated with a single glucose value in the sequence; (3) extracted value of maximal activity level in the sequence; (4) comparing the maximal value to an activity level threshold; and (5) indicating that the glucose anomaly is associated with high activity level, as the maximal stress value surpassed the stress level threshold.

According to some embodiments, the memory may be adapted for storing instructions, for execution by the one or more processors, for: (a) calculating a glucose level ascent rate for the anomaly indicated within the received sequence of glucose level values; (b) comparing the calculated glucose level ascent rate for the anomaly to a predetermined ascent rate threshold; and (c1) indicating that the glucose level anomaly is associated with food intake if the calculated glucose level ascent rate for the anomaly surpasses the predetermined ascent rate threshold, or (c2) indicating that the glucose level anomaly is associated with food intake if the received stress level values remain under the predetermined stress level threshold, the received activity level values remain under the predetermined activity level threshold and the calculated glucose level ascent rate for the anomaly surpasses the predetermined ascent rate threshold.

In FIG. 10A, there is shown a flowchart of a schematic execution example of a decision process for estimating whether a glucose level anomaly is food intake related, in accordance with some embodiments.

In the FIG. 10A example, the following execution steps are exemplified: (1) receiving of a sequence of glucose level values and an indication of anomaly; (2) detecting of a values-ascent within the glucose level values sequence (first 4 values), extracting the minimal and the maximal values along the ascent and calculating the difference between them to receive the ascent range; (3) calculating the ascent duration, in the example: 3 time gaps—between the 4 ascending samples in sequence—with 15 minutes gap between each consecutive sequence samples; (4) calculating the ascent rate; (5) normalizing/scaling of the result; (6) comparing of the normalized ascent rate to an ascent rate threshold; and (7) indicating that the glucose anomaly is associated with food intake, as the calculated ascent rate value surpassed the ascent rate threshold.

According to some embodiments, the memory may be adapted for storing instructions, for execution by the one or more processors, for: (a) calculating a glucose level ascent rate for the anomaly indicated within the received sequence of glucose level values; (b) comparing the calculated glucose level ascent rate for the anomaly to a predetermined ascent rate threshold; and (c1) indicating that the glucose level anomaly is associated with the monitored subject's liver glycogen being broken if the calculated glucose level ascent rate for the anomaly remains under the predetermined ascent rate threshold, or (c2) indicating that the glucose level anomaly is associated with the monitored subject's liver glycogen being broken if the received stress level values remain under the predetermined stress level threshold, the received activity level values remain under the predetermined activity level threshold and the calculated glucose level ascent rate for the anomaly remains under the predetermined ascent rate threshold.

In FIG. 10B, there is shown a flowchart of a schematic execution example of a decision process for estimating whether a glucose level anomaly is liver glycogen being broken related, in accordance with some embodiments.

In the FIG. 10B example, the following execution steps are exemplified: (1) receiving of a sequence of glucose level values and an indication of anomaly; (2) detecting of a values-ascent within the glucose level values sequence (first 4 values), extracting the minimal and the maximal values along the ascent and calculating the difference between them to receive the ascent range; (3) calculating the ascent duration, in the example: 3 time gaps—between the 4 ascending samples in sequence—with 15 minutes gap between each consecutive sequence samples; (4) calculating the ascent rate; (5) normalizing/scaling of the result; (6) comparing of the normalized ascent rate to an ascent rate threshold; and (7) indicating that the glucose anomaly is associated with liver glycogen being broken, as the calculated ascent rate value is under the ascent rate threshold.

According to some embodiments, indicating a glucose anomaly associated cause may include a probability, and/or a level of significance, of the estimated/projected glucose anomaly cause. The probability and/or level of significance of a given glucose anomaly cause estimation/projection may be at least partially based on a combination of characteristics of the detected glucose anomaly/ies and/or other received bio-parameter/condition values, used to generate the estimation/projection, such as: the amplitude/size/range, the length, the frequency, the fluctuation intensity and/or the ascent/descent rates—of the anomaly's/bio-parameter/condition values.

According to some embodiments, the probability and/or level of significance, of a given glucose anomaly cause estimation/projection, may be at least partially based on the extent to which the detected anomaly related data passed, or remained under, the threshold value(s) testing its cause(s). For example, a similar glucose anomaly cause (e.g. stress) may be estimated with a higher probability when the stress indicative values surpassed the stress-test threshold by a relatively greater difference, than when the stress indicative values surpassed the stress-test threshold by a relatively smaller difference (e.g. 20 units delta=90 precent probability and 10 units delta=70 precent probability).

According to some embodiments, indicating a glucose anomaly associated cause may include adding or marking a record, representing the monitored subject's glucose anomaly in the memory or in a functionally associated database, to indicate that the glucose level anomaly is associated with the specific detected cause.

According to some embodiments, records of the system memory, or of a functionally associated database, marked/edited to indicate that the cause of the monitored subject's glucose anomaly as associated with high level of activity, or as associated with the monitored subject's liver glycogen being broken, may be filtered-out of, not included, or removed from a notification que.

According to some embodiments, indicating a glucose anomaly associated cause may include selecting/generating, and relaying by the communication module, of a notification indicating the detected cause that the monitored subject's glucose level anomaly is associated with.

According to some embodiments, the received input sequence of stress level values, may be generated by the system, wherein the communication module is adapted for: (a) receiving a sequence of activity level values, of the monitored subject, sampled concurrently with the sequence of glucose level values; and (b) receiving a sequence of BPM values, of the monitored subject, sampled concurrently with the sequence of glucose level values; and wherein the memory is adapted for storing instructions, for execution by the one or more processors, for: (a) comparing the received activity level values to a predetermined activity level threshold; (b) searching for a BPM values ascent within the received sequence of BPM values; and (c) intermittently registering a high stress level indicative value along a time period in which the received activity level values remained under the predetermined activity level threshold concurrently with a detected ongoing BPM values ascent.

In FIG. 11, there is shown a flowchart of a schematic execution example of a decision process for estimating/determining the stress level of monitored subject, based on the subject's BPM and activity level.

In the FIG. 11 example, the following execution steps are exemplified: (1) receiving of a sequence of BPM values; (2) detecting of a values-ascent within the BPM values sequence (last 4 values); (3) receiving of a sequence of activity level values time corresponding to the sequence of BPM values; (4) extracting the activity level values that are concurrent with the BPM ascent period; (5) comparing each of the activity level values that are concurrent with the BPM ascent period to an activity threshold value; and (6) registering/indicating high stress level values along the BPM ascent period, when ascent value is concurrent with a below threshold activity level value. Estimated/determined stress level values may be provided as system input, to be analyzed, along with a time corresponding glucose level values sequence including an anomaly, to assess whether the glucose anomaly is associated with high stress levels, as described herein.

According to some embodiments, the memory may be further adapted for storing instructions, for execution by the one or more processors, for: (a) populating, for the monitored subject, multiple ‘glucose level anomaly cause’ vectors with respective scores, wherein the score for a given vector, may be at least partially based on a combination of: (i) glucose level anomaly causes' characteristics, for example the frequency of occurrence—over a previous monitoring period—of detected glucose level anomalies caused by a specific anomaly cause represented by the given vector, and/or (ii) one or more subject behavioral or physiological conditions concurrent with detected glucose level anomalies—over a previous monitoring period, wherein the behavioral or physiological conditions are derived/calculated/concluded based on readings/data from a combination of subject monitoring sensors (e.g. composite, wearable, mobile device) and/or subject data provided (e.g. BMI, age, gender, genetic information); and (b) representing the monitored subject over an n-dimensional vector ‘glucose condition’ space, based on the scores of the multiple ‘glucose level anomaly cause’ vectors.

FIG. 7, described hereinbefore, exemplifies the combination of multiple monitored subject related vectors and vector scores—including glucose monitoring and anomaly related, behavioral or physiological condition related, and personal subject data related vectors—to represent/position monitored subjects over an n-dimensional diabetic condition/risk vector space/map.

According to some embodiments, the score for a given ‘glucose level anomaly cause’ related vector, may be based on any combination of multiple detected glucose level anomalies characteristics—over a previous monitoring period—such as: the frequency of occurrence of specific cause associated anomalies, the size/amplitude of specific cause associated anomalies, the time length of specific cause associated anomalies, the ascent and/or descent rate of specific cause associated anomalies and/or other anomaly characteristics.

In FIG. 12A, there is shown a flowchart of a schematic execution example of a decision process for positioning/representing a monitored subject over an n-dimensional vector map/space.

In the FIG. 12A example, the following execution steps are exemplified: (1) the anomaly occurrence frequency breakdown by ‘cause of anomaly’, for subject's detected and registered glucose level anomaly causes over a monitoring period (e.g. a one week monitoring period); and (2) extracted ‘glucose level anomaly cause’ vectors scores, normalized/scaled to a 1-100 scale,—each score indicating subject's position/representation along one dimension/axis within an n-dimensional vector ‘glucose condition’ space—4-dimentional space in the example, as the number of causes assessed.

According to some embodiments, the memory may be further adapted for storing instructions, for execution by the one or more processors, for: (a) populating, for one or more additional monitored subjects, multiple ‘glucose level anomaly cause’ vectors with respective scores and representing/positioning each of the additional monitored subjects over the n-dimensional vector ‘glucose condition’ space; (b) defining high diabetic risk and low diabetic risk regions within the n-dimensional vector ‘glucose condition’ space, wherein regions of multiple relative diabetic risk levels may be defined; (c) selecting one or more of the additional monitored subjects, whose representation over the n-dimensional vector ‘glucose condition’ space is within a region of lower diabetic risk than the diabetic risk within the region in which the monitored subject is currently represented; and (d) generating a monitored subject feedback, including recommendations related to specific vector scores of the monitored subject, to collectively change along the course of time, the vector scores defined representation of the monitored subject over the n-dimensional vector ‘glucose condition’ space, towards the representations of the one or more selected additional monitored subjects whose regions of representation are indicative of lower diabetic risk.

According to some embodiments, defining higher diabetic risk and lower diabetic risk regions within the n-dimensional vector ‘glucose condition’ space, may be based on a combination of: (a) reference of the n-dimensional vector ‘glucose condition’ space representations/positionings, of previously monitored subjects, whose anomalies-cause records/history characteristics are indictive of either high diabetic risk (for example history shows high frequency/magnitude/duration of glucose level anomalies caused by stress and/or food intake), or of low diabetic risk (for example: history shows low frequency/magnitude/duration of glucose level anomalies caused by stress and/or food intake, along with high frequency/magnitude/duration of glucose level anomalies caused by high activity level); (b) reference of the n-dimensional vector ‘glucose condition’ space representations/positionings, of previously monitored subjects, who were otherwise diagnosed as being of high or low diabetic risk, for whom the diagnosis data was received; (c) reference of the n-dimensional vector ‘glucose condition’ space representations/positionings, of previously monitored subjects, for whom information indicating high, or low, diabetic risk (for example: age, gender, BMI, genetic information) was received; and/or (d) reference of the n-dimensional vector ‘glucose condition’ space representations/positionings, of previously monitored subjects, who previously shared the same region/position/cluster over the n-dimensional space as the monitored subject, and checking how many of them are now in better, and how many in a worse, diabetic condition (e.g. developed type 2 diabetes).

According to some embodiments, statistical forecasts, projections and predictions of specific monitored subjects' future diabetic condition/risk (e.g. probability of developing type diabetes) may accordingly be generated, and notified, based on the positions/representations/regions/clusters of multiple subjects over the n-dimensional vector ‘glucose condition’ space.

In FIG. 12B, there is shown a flowchart of a schematic execution example of a decision process for generating/selecting a recommendation for lowering the diabetic risk of a monitored subject, based on the positioning of the monitored subject over an n-dimensional vector ‘diabetic risk map/space, in relation to the positioning of another, one or more, monitored subject who is positioned at a map/space region of lower risk.

In the FIG. 12B example, the following execution steps are exemplified: (1) referencing of ‘glucose level anomaly cause’ vectors scores (normalized/scaled to a 1-100 scale), indicating another subject's, of lower diabetic risk, position/representation within/over the n-dimensional space (4-dimentional, as number of causes assessed, in this example); (2) extracting the largest deltas between same-anomaly-cause vector-scores of the higher and lower diabetic risk subjects; and (3) generating/selecting a recommendation, to lower overall diabetic risk of the monitored subject, relating-to/focusing-on the causes found to have the largest higher-diabetic-risk-subject to lower-diabetic-risk-subject deltas—in the example: ‘work on lowering stress levels and increasing activity levels/durations’.

According to some embodiments, as part of receiving a sequence of glucose level values of a monitored subject, the received sequence may be analyzed by the system to detect glucose levels anomalies within the received values sequence, based on comparison of the received sequence, or of multiple received sequences, to a baseline glucose level pre-generated for the monitored subject.

Accordingly, the communication module may be adapted for: (a) receiving a plurality of value sequences, each sequence including multiple glucose level values of the monitored subject sampled along a course of a specific occurrence of a repeating time period; and (b) receiving an additional value sequence of multiple glucose level values of the monitored subject sampled during a course of a specific following occurrence of the repeating time period; and, the memory may be adapted for storing instructions, for execution by the one or more processors, for: (a) generating, based on the plurality of value sequences, a monitored subject glucose level baseline representing the subject's common glucose levels values along the course of the repeating time period; (b) comparing, for corresponding time segments of the repeating time period, the glucose level values from the additional sequence to the generated subject glucose level baseline values; and (c) determining an anomaly in the monitored subject's glucose level upon comparison results indicating a difference—beyond a predetermined threshold level—between the compared additional sequence values and the generated baseline values.

According to some embodiments, generating, based on the plurality of value sequences, a monitored subject glucose level baseline may include: (a) calculating a measure of central tendency for sets of multiple glucose level values of the monitored subject; wherein each set includes values, from each of the plurality of value sequences, sampled during respective/corresponding/parallel/the-same time segments/points of different occurrences of the repeating time period; and (b) combing multiple measures of central tendency, calculated for various specific time segments/points of the repeating time period, to form a baseline of the monitored subject's common glucose levels values along the course of the repeating time period (e.g. a day).

In FIG. 13, there is shown a flowchart of a schematic execution example, of a decision process for determining anomaly in glucose values, based on the generation of a monitored subject glucose level baseline and the comparison of following glucose level sequences of the monitored subject to the generated baseline.

In the FIG. 13 example, the following execution steps are exemplified: (1) receiving of sequences of glucose level values, of a monitored subject, along specific occurrences of a repeating time period (e.g. the course of a full day); (2) extracting of the subject's common glucose levels values at different time points along the course of the repeating time period, based on a central tendency measure of all glucose levels measured at the same time point/period (e.g. noon time) of the different occurrences of the repeating time period (e.g. a day/24 hours)—in the figure example, the average of the values is being used; (3) receiving of an additional sequence of glucose level values along a following occurrence of the repeating time period; (4) calculating the deltas/differences between the subject's common glucose levels and the time corresponding glucose levels in the additional sequence received; (5) comparing the calculated deltas/differences to a delta/difference threshold (10 in the example); and (6) indicating an anomaly when the delta/difference, between an additional sequence glucose level value and a time corresponding baseline glucose level value, is greater than the threshold—in the example, at the time of the fourth value of the sequences (where the calculated delta is 11.66).

According to some embodiments, various combinations of the systems, components, functions and processes described herein, may be collectively integrated or interconnected to form multi-factor glucose level anomaly cause detection embodiments.

In FIG. 14A, there is shown a flowchart of the steps executed as part of an implementation of a first exemplary ‘multi-factor glucose level anomaly cause detection’ process, based on an integration/interrelation of multiple glucose level anomaly detection functions and processes, as described herein.

In the FIG. 14A example, the following steps are described: (1) receiving a sequence of glucose level values, of a monitored subject, and an indication of an anomaly; (2) checking if anomaly is stress related and indicating; if not, (3) checking if anomaly is activity related and indicating; if not, (4) checking if anomaly is associated with an ascent/decent in glucose level values greater than an ascent/decent range threshold value (e.g. an increase of over 130 mg/dL) and indicating a food intake associated anomaly; if not, (5) checking if anomaly occurred during subject sleep (e.g. based on accelerometer activity, GPS positioning and/or BPM data) and indicating a liver glycogen breakdown associated anomaly; if not, (6) checking if anomaly is associated with an ascent/decent rate in glucose level values greater than an ascent/decent rate threshold value (e.g. an increase rate of over 30 mg/dL per hour) and indicating a food intake associated anomaly, if not, indicating a liver glycogen breakdown associated anomaly.

In FIG. 14B, there is shown a flowchart of the steps executed as part of an implementation of a second exemplary ‘multi-factor glucose level anomaly cause detection’ process, based on an integration/interrelation of multiple glucose level anomaly detection functions and processes, as described herein.

In the FIG. 14B example, the following steps are described: (1) receiving a sequence of glucose level values, of a monitored subject, and an indication of an anomaly; (2) checking if anomaly is activity related and indicating; if not, (3) checking if anomaly is stress related and indicating; if not, (4) checking if anomaly occurred during subject's sleep; if yes, then: (a) checking if anomaly is stress related—if yes indicating anomaly is stress related, if not indicating anomaly is liver glycogen breakdown related; if not, then (b) checking if anomaly's range/change is greater than 30 mg/dL—if yes indicating anomaly is food intake related, if not indicating liver glycogen breakdown related.

Functions, operations, components and/or features described herein with reference to one or more embodiments, may be combined or otherwise utilized with one or more other functions, operations, components and/or features described herein with reference to one or more other embodiments, or vice versa.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A system for glucose level anomaly cause detection, said system comprising:
one or more processors;
a communication module functionally associated with said one or more processors and adapted for:
Receiving a sequence of glucose level values of a monitored subject and an Indication of a glucose levels anomaly within the received values sequence; and
Receiving a sequence of stress level values, of the monitored subject, sampled concurrently with the sequence of glucose level values; and
a memory functionally associated with, and adapted for storing instructions for execution by, said one or more processors, for:
comparing the received stress level values to a predetermined stress level threshold; and
indicating that the glucose level anomaly is associated with a high stress level of the monitored subject if the received stress level values surpass the predetermined stress level threshold.

2. The system according to claim 1, wherein,
said communication module is further adapted for receiving a sequence of activity level values, of the monitored subject, sampled concurrently with the sequence of glucose level values; and wherein,
said memory is further adapted for storing instructions, for execution by said one or more processors, for:
comparing the received activity level values to a predetermined activity level threshold; and
Indicating that the glucose level anomaly is associated with a high activity level of the monitored subject if the received stress level values remain under the predetermined stress level threshold and the received activity level values surpass the predetermined activity level threshold.

3. The system according to claim 2, wherein,
said memory is further adapted for storing instructions, for execution by said one or more processors, for:
calculating a glucose level ascent rate for the anomaly indicated within the received sequence of glucose level values;
comparing the calculated glucose level ascent rate for the anomaly to a predetermined ascent rate threshold;
Indicating that the glucose level anomaly is associated with food intake if the received stress level values remain under the predetermined stress level threshold, the received activity level values remain under the predetermined activity level threshold and the calculated glucose level ascent rate for the anomaly surpasses the predetermined ascent rate threshold; and
Indicating that the glucose level anomaly is associated with the monitored subject's liver glycogen being broken if the received stress level values remain under the predetermined stress level threshold, the received activity level values remain under the predetermined activity level threshold and the calculated glucose level ascent rate for the anomaly remain under the predetermined ascent rate threshold.

4. The system according to claim 3, wherein indicating includes adding or marking a record, representing the monitored subject's glucose anomaly in said memory, to indicate that the glucose level anomaly is associated with the specific detected cause.

5. The system according to claim 4, wherein records of said memory added or marked to indicate that the cause of the monitored subject's glucose anomaly is either, associated with high level of activity, or associated with the monitored subject's liver glycogen being broken, are removed from a notification que.

6. The system according to claim 3, wherein indicating includes selecting/generating, and relaying by said communication module, of a notification indicating the detected cause that the monitored subject's glucose level anomaly is associated with.

7. The system according to claim 1, wherein as part of receiving a sequence of stress level values, said communication module is further adapted for:
receiving a sequence of activity level values, of the monitored subject, sampled concurrently with the sequence of glucose level values; and
receiving a sequence of BPM values, of the monitored subject, sampled concurrently with the sequence of glucose level values; and wherein,
said memory is further adapted for storing instructions, for execution by said one or more processors, for:
comparing the received activity level values to a predetermined activity level threshold;
searching for a BPM values ascent within the received sequence of BPM values; and
intermittently registering a high stress level indicative value along a time period in which the received activity level values remained under the predetermined activity level threshold concurrently with a detected ongoing BPM values ascent.

8. The system according to claim 3, wherein, said memory is further adapted for storing instructions, for execution by said one or more processors, for:
populating, for the monitored subject, multiple ‘glucose level anomaly cause’ vectors with respective scores, wherein the score for a given ‘glucose level anomaly cause’ vector, is at least partially based on the frequency of occurrence—over a previous monitoring period—of detected glucose level anomalies caused by a specific anomaly cause represented by the given ‘glucose level anomaly cause’ vector; and
representing the monitored subject over an n-dimensional vector ‘glucose condition’ space, based on the scores of the multiple ‘glucose level anomaly cause’ vectors.

9. The system according to claim 8, wherein, said memory is further adapted for storing instructions, for execution by said one or more processors, for:
populating, for one or more additional monitored subjects, multiple ‘glucose level anomaly cause’ vectors with respective scores and representing each of the additional monitored subjects over the n-dimensional vector ‘glucose condition’ space;
defining higher diabetic risk and lower diabetic risk regions within the n-dimensional vector ‘glucose condition’ space;
selecting one or more of the additional monitored subjects, whose representation over the n-dimensional vector ‘glucose condition’ space is within a region of lower diabetic risk than the diabetic risk within the region in which the monitored subject is currently represented; and
generating a monitored subject feedback, including recommendations related to specific vector scores of the monitored subject, to collectively change along time, the vector scores defined representation of the monitored subject over the n-dimensional vector ‘glucose condition’ space, towards the representations of the one or more selected additional monitored subjects whose regions of representation are indicative of lower diabetic risk.

10. The system according to claim 1, wherein as part of receiving a sequence of glucose level values of a monitored subject and an indication of a glucose levels anomaly within the received values sequence, said communication module is further adapted for:
receiving a plurality of value sequences, each sequence including multiple glucose level values of the monitored subject sampled along a course of a specific occurrence of a repeating time period; and
receiving an additional value sequence of multiple glucose level values of the monitored subject sampled during a course of a specific following occurrence of the repeating time period; and wherein,
said memory is further adapted for storing instructions, for execution by said one or more processors, for:
generating, based on the plurality of value sequences, a monitored subject glucose level baseline representing the subject's common glucose levels values along the course of the repeating time period;
comparing, for corresponding time segments of the repeating time period, the glucose level values from the additional sequence to the generated subject glucose level baseline values; and
determining an anomaly in the monitored subject's glucose level upon comparison results indicating a difference—beyond a predetermined threshold level—between the compared additional sequence values and the generated baseline values.

11. A method for glucose level anomaly cause detection, said method comprising:
receiving a sequence of glucose level values of a monitored subject and an Indication of a glucose levels anomaly within the received values sequence;
receiving a sequence of stress level values, of the monitored subject, sampled concurrently with the sequence of glucose level values;
comparing the received stress level values to a predetermined stress level threshold; and
Indicating that the glucose level anomaly is associated with a high stress level of the monitored subject if the received stress level values surpass the predetermined stress level threshold.

12. The method according to claim 11, further comprising:
receiving a sequence of activity level values, of the monitored subject, sampled concurrently with the sequence of glucose level values;
comparing the received activity level values to a predetermined activity level threshold; and
Indicating that the glucose level anomaly is associated with a high activity level of the monitored subject if the received stress level values remain under the predetermined stress level threshold and the received activity level values surpass the predetermined activity level threshold.

13. The method according to claim 12, further comprising:
calculating a glucose level ascent rate for the anomaly indicated within the received sequence of glucose level values;
comparing the calculated glucose level ascent rate for the anomaly to a predetermined ascent rate threshold;
indicating that the glucose level anomaly is associated with food intake if the received stress level values remain under the predetermined stress level threshold, the received activity level values remain under the predetermined activity level threshold and the calculated glucose level ascent rate for the anomaly surpasses the predetermined ascent rate threshold; and
indicating that the glucose level anomaly is associated with the monitored subject's liver glycogen being broken if the received stress level values remain under the predetermined stress level threshold, the received activity level values remain under the predetermined activity level threshold and the calculated glucose level ascent rate for the anomaly remain under the predetermined ascent rate threshold.

14. The method according to claim 13, wherein indicating includes marking/labeling/writing-to a database record representing the monitored subject's glucose anomaly, that the glucose level anomaly is associated with the specific detected cause.

15. The method according to claim 14, wherein database records marked/labelled/written-to to indicate that the cause of the monitored subject's glucose anomaly is either, associated with high level of activity, or associated with the monitored subject's liver glycogen being broken, are removed from a notification que.

16. The method according to claim 13, wherein indicating includes selecting or generating and relaying a notification indicating the detected cause that the monitored subject's glucose level anomaly is associated with.

17. The method according to claim 11, wherein receiving a sequence of stress level values, of the monitored subject, includes a preprocess of:
receiving a sequence of activity level values, of the monitored subject, sampled concurrently with the sequence of glucose level values;
comparing the received activity level values to a predetermined activity level threshold;
receiving a sequence of BPM values, of the monitored subject, sampled concurrently with the sequence of glucose level values;
searching for a BPM values ascent within the received sequence of BPM values; and
intermittently registering a high stress level value along a time period in which the received activity level values remained under the predetermined activity level threshold concurrently with a detected ongoing BPM values ascent.

18. The method according to claim 13, further including:
populating, for the monitored subject, multiple ‘glucose level anomaly cause’ vectors with respective scores, wherein the score for a given ‘glucose level anomaly cause’ vector, is at least partially based on the frequency of occurrence—over a previous monitoring period—of detected glucose level anomalies caused by a specific anomaly cause represented by the given ‘glucose level anomaly cause’ vector; and
representing the monitored subject over an n-dimensional vector ‘glucose condition’ space, based on the scores of the multiple ‘glucose level anomaly cause’ vectors.

19. The method according to claim 18, further including:
populating, for one or more additional monitored subjects, multiple ‘glucose level anomaly cause’ vectors with respective scores and representing each of the additional monitored subjects over the n-dimensional vector ‘glucose condition’ space;
defining higher diabetic risk and lower diabetic risk regions within the n-dimensional vector ‘glucose condition’ space;
selecting one or more of the additional monitored subjects, whose representation over the n-dimensional vector ‘glucose condition’ space is within a region of lower diabetic risk than the diabetic risk within the region in which the monitored subject is currently represented; and
generating a monitored subject feedback, including recommendations related to specific vector scores of the monitored subject, to collectively change, along time, the vector scores defined representation of the monitored subject over the n-dimensional vector ‘glucose condition’ space, towards the representations of the one or more selected additional monitored subjects whose regions of representation are indicative of lower diabetic risk.

20. The method according to claim 11, wherein receiving a sequence of glucose level values of a monitored subject and an indication of a glucose levels anomaly within the received values sequence, includes detecting the anomaly by:
receiving a plurality of value sequences, each sequence including multiple glucose level values of the monitored subject sampled along a course of a specific occurrence of a repeating time period;
generating, based on the plurality of value sequences, a monitored subject glucose level baseline representing the subject's common glucose levels values along the course of the repeating time period;
receiving an additional value sequence of multiple glucose level values of the monitored subject sampled during a course of a specific following occurrence of the repeating time period;
comparing, for corresponding time segments of the repeating time period, the glucose level values from the additional sequence to the generated subject glucose level baseline values; and
determining an anomaly in the monitored subject's glucose level upon comparison results indicating a difference—beyond a predetermined threshold level—between the compared additional sequence values and the generated baseline values.
